(19) United States
(12) Patent Application Publication
(10) Pub. No.: US 2023/0190205 A1
(43) Pub. Date: Jun. 22, 2023

(54) MOBILITY LEVEL USER INTERFACE INCLUDING SMART TURN REMINDERS BASED ON RISK OF PATIENT

(71) Applicant: Hill Rom Services, Inc., Batesville, IN (US)

(72) Inventors: Timothy Receveur, Apex, NC (US); Sinan Batman, Cary, NC (US); Eugene Urrutia, Apex, NC (US); Hyeon Ki Jeong, Atlanta, GA (US)

(21) Appl. No.: 18/068,142

(22) Filed: Dec. 19, 2022

Related U.S. Application Data
(60) Provisional application No. 63/292,119, filed on Dec. 21, 2021.

(51) Int. Cl. A61B 5/00; A61B 5/11

(52) U.S. Cl. CPC A61B 5/7264; A61B 5/6892; A61B 5/742; A61B 5/7475; A61B 5/1116; A61B 5/1113; A61B 2562/0252; A61B 2562/046

(57) ABSTRACT

A patient support apparatus comprises a plurality of load cells, a frame supported on the load cells, a mattress, a plurality of air pressure sensors, and a control system. The mattress includes a plurality of inflatable zones positioned on the frame, the mattress and frame cooperating to direct any patient load through the mattress and frame to the load cells. Each of the plurality of air pressure sensors measures the pressure in a respective inflatable zone of the mattress. The control system includes a controller operable to receive a separate signal from each of the plurality of load cells and each of the plurality of air pressure sensors and process the signals to identify movement data and communicate the movement data to a caregiver.

### PRIORITY CLAIM

This application claims priority under 35 U.S.C. § 119(e) to U.S. Provisional Application No. 63/292,119, filed Dec. 21, 2021, which is expressly incorporated by reference herein.

### BACKGROUND

The present disclosure relates to the use of sensors in a patient support apparatus, such as a hospital bed, for example, to detect patient motion and characterize the patient motion. More specifically, the present disclosure is directed to a system or method for communicating information concerning the patient's mobility to a caregiver.

The use of load cells in patient support apparatuses, such as hospital beds, for example, to measure patient weight is known. Over time, approaches to using the information from the load cells to detect patient movement and to issue an alert or notification when the patient moves beyond a particular threshold have been developed. The use of load cells to make these determinations and inferences based on the motion or movement is limited by the potential for external influences, such as by the addition of equipment to the frame supported on the scale. When this is done, the existing information regarding the position of the patient is compromised as the weight distribution is changed unexpectedly.

Pressure sensors used to measure air pressure in zones of an inflatable mattress are used to control the inflation pressure in the zones and to control the interface pressure experienced by a patient supported on the mattress. However, because of transient effects and lack of precision, air pressure sensors associated with mattress zones are not regularly used to measure patient information. In addition, caregivers or visitors may intermittently apply pressure to the mattress, thereby changing air pressure measurements and the distribution of the weight on the frame. Motion algorithms generally rely on changes in the distribution of weight over multiple sensors to determine patient location and relative movement. These transient and external forces confound the algorithms used to determine patient movement and motion.

In some cases, it is important to determine patient movement relative to the patient support apparatus. Movement in this context means a change in position of the patient on the patient support apparatus, such as rolling over or moving toward an edge of the patient support apparatus in order to exit the patient support apparatus. The patient's mobility including information about when the patient was turned may be indicative of a patient's health and/or medical needs.

The caregiver may need to move or turn the patient intermittently depending on the patient condition and needs. The caregiver may need to keep track of the time between any required patient movements. It may also be important that the caregiver is aware of the overall improvement or decline of the patient's health so that they can assess how much time and attention the patient needs. In addition, the caregiver may also want to know how to respond to the mobility information and may need direction about the care the patient needs. Thus, a patient's mobility information should be reported in a manner that provides the required information including patient turn data to the caregiver and allows the caregiver to provide optimal care for the patient.

### SUMMARY

The present disclosure includes one or more of the features recited in the appended claims and/or the following features which, alone or in any combination, may comprise patentable subject matter.

According to a first aspect of the present disclosure, a patient support apparatus comprises a control system and a user interface. The control system includes a controller operable to detect and classify movement data. The user interface includes a display illustrating the patient movement data. The movement data comprises patient movement data and non-patient motion artifacts.

In some embodiments of the first aspect, the display comprises a first portion and a second portion.

In some embodiments of the first aspect, the patient movement data comprises patient mobility score based on patient movement amplitude and patient frequency.

In some embodiments of the first aspect, the patient movement data comprises slight movement or major movement.

In some embodiments of the first aspect, the patient mobility score is displayed on the user interface as a mobility trend-line on the first portion of the display.

In some embodiments of the first aspect, the patient movement data displayed on the user interface comprises identification of caregiver assisted motion on the second portion of the display.

In some embodiments of the first aspect, the caregiver assisted motion occurs when both non-patient motion artifact and patient movement occurs at the same time, and when the non-patient motion artifact comprises more than about 20% of the movement data at that time.

In some embodiments of the first aspect, the patient movement amplitude comprises slight movement when the patient movement does not comprise a postural change for a period of about 30 seconds.

In some embodiments of the first aspect, the patient movement amplitude comprises major movement when the patient movement comprises a postural change in a period of about 30 seconds.

In some embodiments of the first aspect, a caregiver inputs a turning protocol comprising intervening time between patient turns, and wherein the patient movement data displayed on the user interface comprises a color coded display of the current time in relationship to the turning protocol.

In some embodiments of the first aspect, the turning protocol is also affected by an amount of major movements or slight movements of the patient.

In some embodiments of the first aspect, the patient movement is monitored by one or more continuous sensors.

According a second aspect of the present disclosure, a system comprises a patient support surface supporting a patient, a controller operable to detect and classify patient movement, and a user interface including a display, the display illustrating the patient movement data, wherein movement data comprises patient movement data and non-patient motion artifacts.

In some embodiments of the second aspect, the display comprises a first portion and a second portion.

In some embodiments of the second aspect, the patient movement data comprises patient mobility score based on patient movement amplitude and patient frequency.

In some embodiments of the second aspect, the patient movement data comprises slight movement or major movement.

In some embodiments of the second aspect, the patient mobility score is displayed on the user interface as a mobility trend-line on the first portion of the display.

In some embodiments of the second aspect, the patient movement data displayed on the user interface comprises identification of caregiver assisted motion on the second portion of the display.

In some embodiments of the second aspect, the caregiver assisted motion occurs when both non-patient motion artifact and patient movement occurs at the same time, and when the non-patient motion artifact comprises more than about 20% of the movement data at that time.

In some embodiments of the second aspect, the patient movement amplitude comprises major movement when the patient movement comprises a postural change in a period of about 30 seconds.

In some embodiments of the second aspect, the patient movement amplitude comprises slight movement when the patient movement does not comprise a postural change for a period of about 30 seconds.

In some embodiments of the second aspect, a caregiver inputs a turning protocol comprising intervening time between patient turns, and wherein the patient movement data displayed on the user interface comprises a color coded display of the current time in relationship to the turning protocol.

In some embodiments of the second aspect, the turning protocol is also affected by an amount of major movements or slight movements of the patient.

In some embodiments of the second aspect, the patient movement is monitored by one or more continuous sensors.

According to a third aspect of the present disclosure, a method of displaying movement data collected from a support apparatus comprising an inflatable mattress includes the steps of: monitoring signals from a plurality of load cells supporting an inflatable mattress, using a controller to process the signals from the load cells to detect and classify movement data, and displaying the movement data on a display on a user interface, wherein the movement data comprises patient movement data and non-patient motion artifacts.

In some embodiments of the third aspect, the display comprises a first portion and a second portion.

In some embodiments of the third aspect, the patient movement data comprises patient mobility score based on patient movement amplitude and patient frequency.

In some embodiments of the third aspect, the patient movement data comprises slight movement or major movement.

In some embodiments of the third aspect, the patient mobility score is displayed on the user interface as a mobility trend-line on the first portion of the display.

In some embodiments of the third aspect, the patient movement data displayed on the user interface comprises identification of caregiver assisted motion on the second portion of the display.

In some embodiments of the third aspect, the caregiver assisted motion occurs when both non-patient motion artifact and patient movement occurs at the same time, and when the non-patient motion artifact comprises more than about 20% of the movement data at that time.

In some embodiments of the third aspect, the patient movement amplitude comprises major movement when the patient movement comprises a postural change in a period of about 30 seconds.

In some embodiments of the third aspect, the patient movement amplitude comprises slight movement when the patient movement does not comprise a postural change for a period of about 30 seconds.

In some embodiments of the third aspect, the method further comprises a caregiver inputting a turning protocol comprising intervening time between patient turns, and wherein the patient movement data displayed on the user interface comprises a color coded display of the current time in relationship to the turning protocol.

In some embodiments of the third aspect, the turning protocol is also affected by an amount of major movements or slight movements of the patient.

In some embodiments of the third aspect, the patient movement is monitored by one or more continuous sensors.

Additional features, which alone or in combination with any other feature(s), such as those listed above and/or those listed in the claims, can comprise patentable subject matter and will become apparent to those skilled in the art upon consideration of the following detailed description of various embodiments exemplifying the best mode of carrying out the embodiments as presently perceived.

### BRIEF DESCRIPTION OF THE DRAWINGS

The detailed description particularly refers to the accompanying figures in which:

FIG. 1 is a perspective view of a patient support apparatus including a control system operable to measure signals from a plurality of sensors and process those signals according to the present disclosure;

FIG. 2 is a block diagram of a portion of the control system of the patient support apparatus of FIG. 1;

FIG. 3 is a diagrammatic illustration of the interaction between a first frame of the patient support apparatus of FIG. 1 and a second frame supported on load cells supported from the first frame;

FIG. 4, is a side view of a portion of the patient support apparatus of FIG. 1 showing a first frame supported on load cells supported on a second frame, the load cells supporting all of the load of the first frame;

FIG. 5 is an illustration of one embodiment of a user interface displaying a patient's mobility information; and

FIG. 6 illustrates a rule-based algorithm implemented to determine a patient mobility score of a patient on the patient support apparatus.

### DETAILED DESCRIPTION

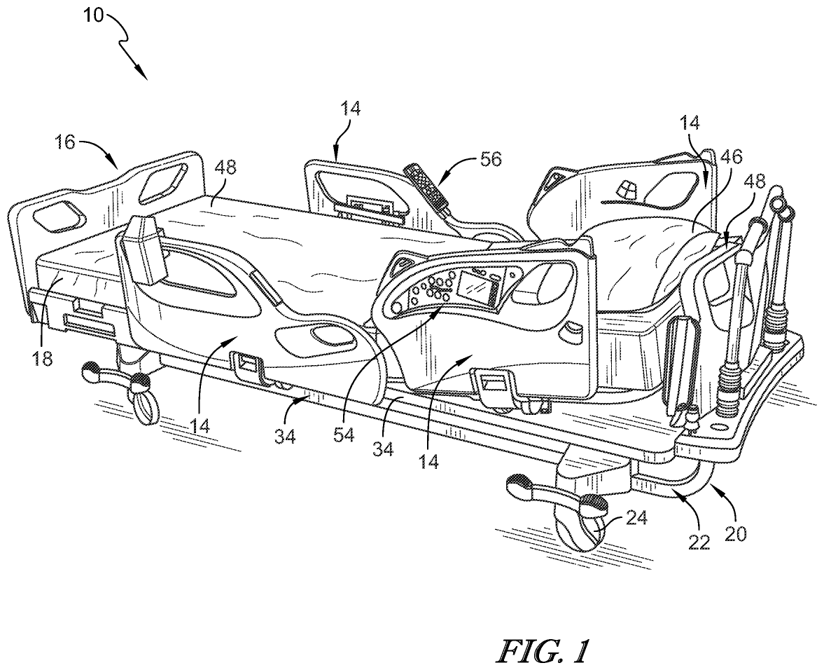

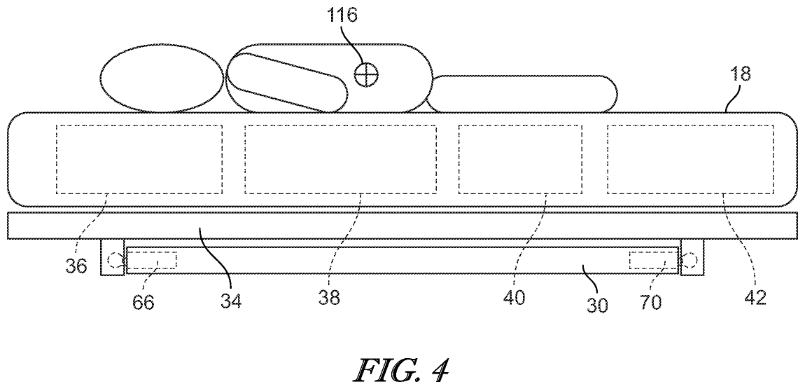

An illustrative patient support apparatus 10 embodied as a hospital bed is shown in FIG. 1. The bed 10 of FIG. 1 has a frame 20 which includes a base frame 22 supported on casters 24. The stationary base frame 22 further supports a weigh frame 30 that an adjustably positionable mattress support upper frame 34 supporting a mattress 18. As shown in FIG. 4, the illustrative mattress 18 is an inflatable patient support surface which includes inflatable zones including a head zone 36, a seat zone 38, thigh zone 40, and a foot zone 42. The bed 10 further includes a headboard 12 at a head end 46 of the bed 10, a footboard 16 at a foot end 48 of the bed 10, and a movable siderails 14 coupled to the upper frame 34 of the bed 10. The bed 10 also includes a user interface 54 positioned on one of the siderails 14. The bed 10 of the embodiment of FIG. 1 is conventionally configured to adjustably position the upper frame 34 relative to the base frame 22 to adjust the position of a patient supported on the mattress 18.

Conventional structures and devices may be provided to adjustably position the upper frame 34, and such conventional structures and devices may include, for example, linkages, drives, and other movement members and devices coupled between base frame 22 and the weigh frame 30, and/or between weigh frame 30 and upper frame 34. Control of the position of the upper frame 34 and mattress 18 relative to the base frame 22 or weigh frame 30 is controlled, for example, by a patient control pendant 56 or user interface 54. The upper frame 34 may, for example, be adjustably positioned in a general incline from the head end 46 to the foot end 48 or vice versa. Additionally, the upper frame 34 may be adjustably positioned such that the head section 44 of the mattress 18 is positioned between minimum and maximum incline angles, e.g., 0-65 degrees, relative to horizontal or bed flat, and the upper frame 34 may also be adjustably positioned such that a seat section (not shown) of the mattress 18 is positioned between minimum and maximum bend angles, e.g., 0-35 degrees, relative to horizontal or bed flat. Those skilled in the art will recognize that the upper frame 34 or portions thereof may be adjustably positioned in other orientations, and such other orientations are contemplated by this disclosure.

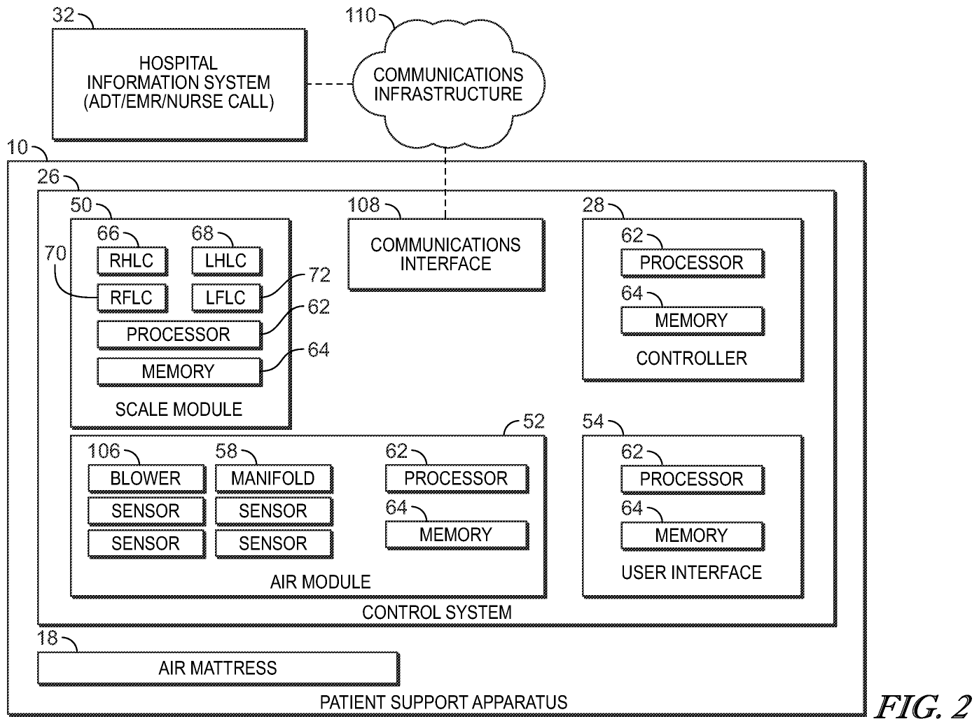

In one illustrative embodiment shown diagrammatically in FIG. 2, the bed 10 has a control system 26 that includes a controller 28, a scale module 50, an air module 52, and the user interface 54. In the illustrative embodiment each of the controller 28, scale module 50, air module 52, and user interface 54 includes a processor 62 and a memory device 64. The processor 62 and memory device 64 are shown only with respect to the controller 28, but similar structures are used in the scale module 50, air module 52, and user interface 54. The memory device 64 includes instructions that, when executed by the processor 62, cause the processor 62 to perform functions as associated with the particular one of controller 28, scale module 50, air module 52, and user interface 54. The components of the control system 26 communicate amongst themselves to share information and distribute the functions of the bed 10. The processor 62 of each of the controller 28, scale module 50, air module 52, and user interface 54 is also operable, based on instructions from the memory device 64, to communicate with the others of the controller 28, scale module 50, air module 52, and user interface 54 using a communications protocol. It should be understood that the term processor here includes any microprocessor, microcontroller, processor circuitry, control circuitry, preprogrammed device, or any structure capable of accessing the memory device and executing non-transient instructions to perform the tasks, algorithm, and processed disclosed herein. In the illustrative embodiment, the control system 26 employs a conventional controller area network (CAN) for communications between subsystems, but it should be understood that any of a number of networking and communications solutions could be employed in the control system 26.

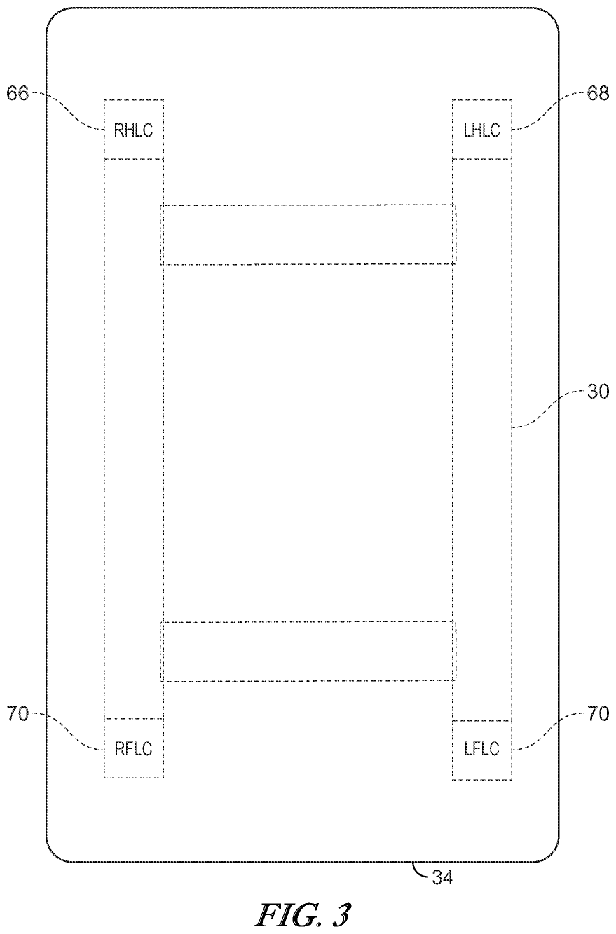

As shown in FIG. 3, the scale module 50 includes four load cells 66, 68, 70, and 72. To determine a weight of a patient supported on the mattress 18, the load cells 66, 68, 70, and 72 are positioned between the weigh frame 30 and the upper frame 34 as illustrated in FIGS. 3 and 4. Each load cell 66, 68, 70, 72 is configured to produce a signal indicative of a load supported by the respective load cell 66, 68, 70, 72 from the upper frame 34 relative to the weigh frame 30. Some of the structural components of the bed 10 will be designated hereinafter as “right”, “left”, “head” and “foot” from the reference point of an individual lying on the individual's back on the mattress 18 with the individual's head oriented toward the head end 46 of the bed 10 and the individual's feet oriented toward the foot end 48 of the bed 10. Following this convention, the load cell 66 is designated as the right head load cell (RHLC) in the figures to represent that the load cell 66 is positioned at the right side of the bed 10 at the head end 46. The load cell 68 is designated at the left head load cell (LHLC), the load cell 70 is designated as the right foot load cell (RFLC), and the load cell 72 is designated left foot load cell (LFLC), each following the same convention.

The scale module 50 includes a processor 62 that is in communication with each of the respective load cells 66, 68, 70, and 72 and operable to process the signals from the load cells 66, 68, 70, and 72. The memory device 64 is also utilized by the controller 28 to store information corresponding to features and functions provided by the bed 10.

A weight distribution of a load among the plurality of load cells 66, 68, 70, and 72 may not be the same depending on variations in the structure of the bed 10, variations in each of load cells 66, 68, 70, and 72 and the position of the load on the mattress 18 relative to the particular load cell 66, 68, 70, or 72. Accordingly, a calibration constant for each of the load cells 66, 68, 70, and 72 is established to adjust for differences in the load cells 66, 68, 70, and 72 in response to the load borne by each. Each of the load cells 66, 68, 70, and 72 produces a signal indicative of the load supported by that load cell 66, 68, 70, or 72. The loads detected by each of the respective load cells 66, 68, 70, 72 are adjusted using a corresponding calibration constant for the respective load cell 66, 68, 70, 72. The adjusted loads are then combined to establish the actual weight supported on the bed 10. In the present disclosure, the independent signals from each of the load cells 66, 68, 70, 72 is used to draw inferences about the movement and motion of the patient.

The air module 52 is the functional controller for the mattress 18 and includes processor 62 and a memory device 64. The processor 62 is in communication with a blower 106, a manifold 58, and an air pressure sensor assembly 60. The air module 52 is a conventional structure with the manifold 58 operating under the control of the processor 62 to control the flow of air from the blower 106 into and out of the head zone 36, seat zone 38, thigh zone 40, and foot zone 42 to control the interface pressure experienced by the patient supported on the mattress 18. The sensor assembly 60 includes separate sensors for measuring the air pressure in each of the head zone 36, seat zone 38, thigh zone 40, and foot zone 42. The pressure sensor assembly includes a head zone sensor 82, a seat zone sensor 84, a thigh zone senor 86, and a foot zone sensor 88. While signals from the sensors 82, 84, 86, and 88 are used to control the pressure in the respective zones, applying the principles of the present disclosure, the signals are also useful in making inferences regarding patient movement and, when used synergistically with the information gleaned from the signals from the load cells 66, 68, 70, and 72, provide a more fulsome and accurate analysis of patient movement and/or any motion associated with the patient support apparatus.

The scale module 50 and air module 52 of the bed 10 are used for measuring the motions of a patient that occupies the bed 10. Referring to FIG. 4, a diagrammatic side view of a patient supported on the mattress 18 and frame 34 with the load of the patient being borne by the inflated zones 36, 38, 40, and 42 and passed through the mattress to the frame 34 to the load cells 66, 68, 70, and 72 supported from the weigh frame 30. As seen in FIG. 4, in a static condition, the patient's weight is appropriately distributed over the inflated zones 36, 38, 40, and 42. Each of those zones 36, 38, 40, and 42 are inflated to a target pressure based on the patient's weight detected by the scale module 50 and the expected distribution of the patient.

Through an empirical study that included real-time data collection from video observation of test subject patients synchronized with signals from load cells of the scale module of the bed supporting the test subject patients, the types of motion from the where classified in one of three types: lateral patient motions (LPMs); vertical or self-offloading patient movements (SOs); or non-patient motion artifacts (NPMAs). There were also observations that found that load cell signals varied when there was no patient movement. These artifacts were designated as non-movements (NMs). Permutations of these categories, called “complex movements”, also including further categorization into combinations including different directionality of the simple movements was also established.

Once the motion is identified as a patient movement or a non-patient movement, the patient movements are aggregated into events and the degree of motion is classified. The degree of motion may be major patient motion, slight patient motion, and/or nurse-assisted motion. A motion classifier may be used to assess patient movement to determine the degree of motion and then uses the identified degree of motion and motion frequency to determine a patient mobility score.

In one embodiment, the determination of major patient movement vs slight patient movement vs caregiver or nurse assisted patient movement motion may be based on the caregiver or nurse assessment of the Braden score and/or Braden mobility sub-score of the aggregated motions. The mobility subscale of the Braden scale defines the four mobility levels. Completely immobile is defined as when the subject does not make even slight changes in body or extremity position without assistance. Very limited movement is defined as when the subject makes occasional slight changes in body or extremity position but unable to make frequent or significant changes independently. Slightly limited movement is defined as when the subject makes frequent though slight changes in body or extremity position independently. No limitation is defined as when the subject makes major and frequent changes in position without assistance. Thus, mobility may be characterized by movement amplitude (slight or major movement) and movement frequency (number of movements per hour ranging between no movement, occasional movement and frequent movement). The patient movement amplitude comprises major movement when the patient movement comprises a postural change in a period of about 30 seconds

In some embodiments, one or more continuous sensors may be used to monitor patient movement. The one or more continuous sensors used to monitor patient movement may capture more information than a nurse or a caregiver coming several times in to a patient room. In some embodiments, movement monitored by the one or more continuous sensors may be used and processed by the controller 28. In one embodiment, a rule-based algorithm illustrated may be implemented to determine a patient mobility score of a patient on bed 10. In some embodiments, a threshold based on patient sleep activity may be used to correspond to the extreme minimal value of patient movement may be appropriate for a patient who is in pain or is dazed with medication.

The present disclosure is directed to a system and method for communicating a movement data identified on the patient support apparatus 10 to a caregiver after the movement data has been determined by the controller 28. In one embodiment, the movement data identified on the patient support apparatus 10 is communicated to a caregiver through a user interface 54 with one or more displays. In other embodiments, the movement data identified on the patient support apparatus 10 is used to determine when the patient was turned and how the patient's turn history relates to patient care.

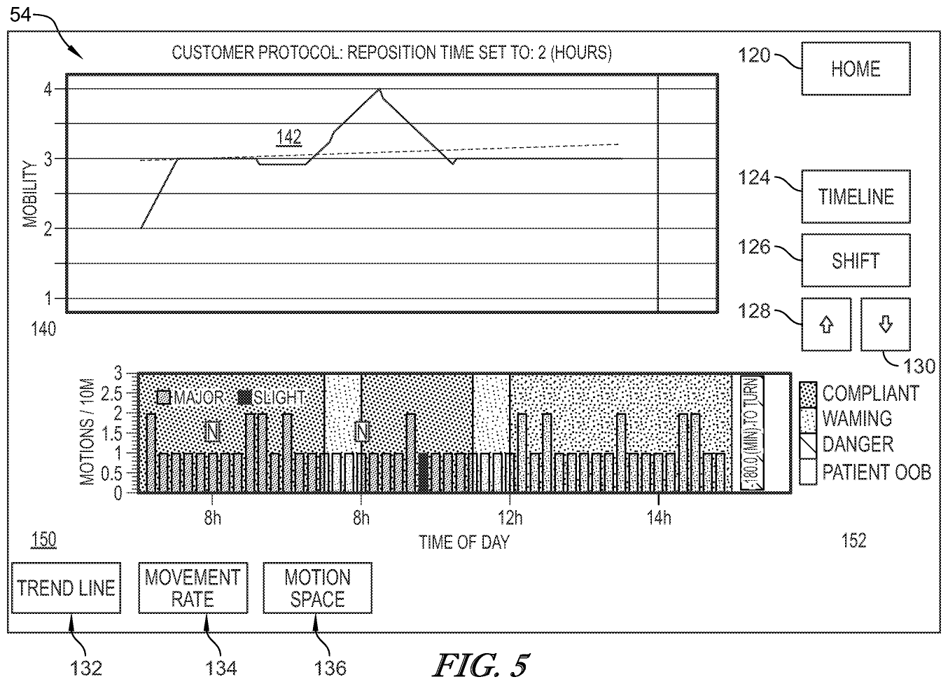

FIG. 5 illustrates the user interface 54 which includes a display screen 140, a home button 120, a timeline button 124, a shift button 126, a up button 128, down arrow button 130, a trend line button 132, a movement rate button 134, and a motion space button 136.

In one embodiment, a patient's mobility is determined based on the amplitude and frequency of patient movement. The patient's movement is classified as a slight movement of major movement, and the patient is assigned a mobility score (e.g., 1, 2, 3, 4) based on the number of slight and major movement in a given time period.

In one embodiment, a patient's mobility score is calculated on an hourly basis, and a linear trend line 142 is displayed on the display screen 140 when the caregiver selects a trend line button 132. The trend line 142 may be color coded to match the upward (e.g., green) or downward (e.g., red) trend. The trend line may be displayed for a given timeline. The timeline may comprise one or more days, or be based on the number of hour long shifts of the nurse or caregiver. In one embodiment, the caregiver may determine the timeline by using the timeline button 124. The timeline button 124 may determine the number of day comprising the trend line 142. In other embodiments, the caregiver may determine the timeline by using the shifts button 126. The shifts button 126 may determine the number of hour long shifts comprising the trend line 142.

In one embodiment, as illustrated in FIG. 5, the user interface 54 includes a second display screen 150 that illustrates the minor or slight movement rates. The minor or slight movement rates may be illustrated as function of the number of minor or slight movement rates measured per unit time. For example, the second display screen 150 may illustrate the number of minor of slight movements measured every 10 minute interval when the caregiver uses the movement rate button 134. The caregiver may also be able to determine the time interval used to calculate the movement rate by using the movement rate button 134. The user interface 54 may include an icon “N” in the second display 150. The icon “N” indicates that a caregiver or nurse has repositioned the patient or indicates nurse-assisted movement at the time corresponding to the icon “N” of the second display 150. In some embodiments, the icon “N” may be replaced by an icon of a person moving another person, or a “nurse face” to denote that a caregiver or nurse may have assisted in patient movement. In one embodiment, the icon “N” or caregiver or nurse-assisted movement may detected because a bed's turn assist function was evoked. In some embodiments, the icon “N” or caregiver or nurse-assisted movement may be detected by the controller 28 based on the data processed by the processor 62.

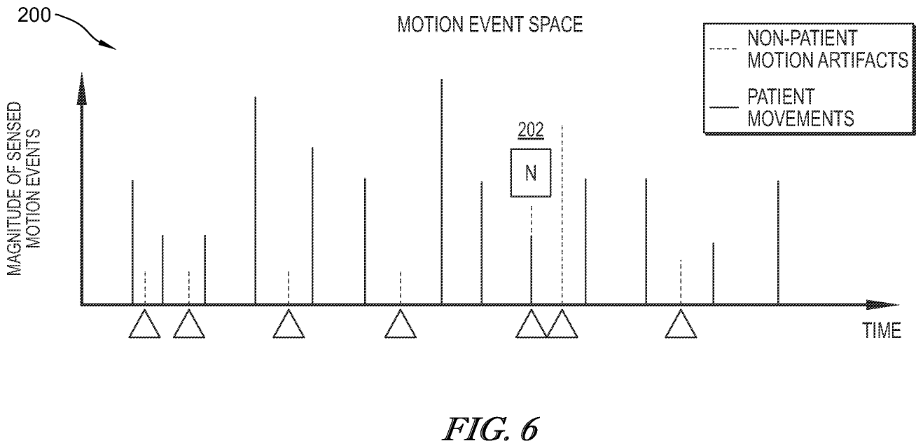

In one embodiment, motions due to non-patients movement and patient movement is tracked through time as shown in FIG. 6. The magnitude of the motion is shown on the y-axis and may vary with each motion. The motion due to patient movement and non-patient movement may coincide in time as indicated by 202. If the motion due to patient movement and non-patient movement coincides in time, the controller 28, based on the data processed by the processor 62, may determine that a caregiver or nurse assisted motion occurred that time. The motion event space 200 may be shown on the first display 140 or on the second display 150 when the caregiver or nurse accesses the motion space button 136.

The types of patient motion can be classified into one of three types: lateral patient motions (LPMs); vertical or self-offloading patient movements (SOs); or non-patient motion artifacts (NPMAs). Additionally, signals from load cell or other sensors on the bed 10 can vary when there are no patient movement. These artifacts are designated as non-movements (NMs). Permutations of these categories, called “complex movements”, also including further categorization into combinations including different directionality of the simple movements can also be established.

Various tools and methodologies may be used to determine if a caregiver or nurse assisted motion occurred at any given time point. In one embodiment, load cell signal data that has aspects of NPMA movement can be determined using the algorithm outlined in U.S. Provisional Application 62/238,817 titled “Patient Mobility Classification”, which is incorporated by reference herein for the disclosure of methods of determining patient mobility.

Signals from the load cells of a bed, such as load cells 66, 68, 70, and 72 can be used for the detection of lateral patient motions (LPMs), which are, by definition, detected patient motions which have no vertical component. Any lateral movement will cause the center of gravity of the load supported on the bed 10 to change during a unit-time interval, proportionally to ratio of the displacement of the amount of mass moved to the amount of mass that remained stationary.

Different approaches may be used to discriminate patient motion types. If there is no total gain of loads, and the loads simply shift around the four load cells 66, 68, 70, and 72 as the patient moves laterally, the motion is an LPM. In contrast, when a caregiver pushes or pulls on the patient or bed 10 and the net load on the load cells 66, 68, 70, and 72 is increased or decreased, the motion is an NPMA.

This is the case for both transient touches of the bed 10, such as when a person hugs the patient, and in sustained touches of the bed 10, such as when a caregiver leans on bed 10 while doing long procedure. In either case, an additional load is introduced to the load cells 66, 68, 70, and 72 resulting in a material change from the sum of the loads on each load cell 66, 68, 70, and 72 when the transient load is applied to the bed 10.

The value of a transient load, designated as total transient load (TTL) is calculated by subtracting the from the total load measured by the load cells 66, 68, 70, and 72 the closed-system load measured before the transient event; the closed-system load which is effectively the patient's static weight, designated as the DC sum of beams (DCSB) which can be determined using known techniques, such as that disclosed in U.S. Pat. No. 10,054,479 titled “BED WITH AUTOMATIC WEIGHT OFFSET DETECTION AND MODIFICATION,” which is incorporated herein for the disclosure of monitoring and updating a patient load to establish a static patient weight, DCSB.

Once the DCSB is established, a simple threshold can be tested to determine whether a TTL is a NPMA or not. An oscillation in the location of the center of gravity of the weight supported on the bed 10 and an effective return of the TTL to zero can be considered to confirm the transient nature of the load to help confirm that the event is a TTL. If a TTL is a NPMA it may be plotted in a different color (e.g., green) than a patient movement (e.g., blue) in the motion event space. Alternatively, they may be plotted with different visual indications as shown in FIG. 6

In one embodiment, relying just on thresholding TTL moment-by-moment may be confounded by self-offloading patient movements (SOs). SOs are large vertical shifts that are an artifact of a patient quickly lifting their core body up using the strength in his legs or arms and then returning to a starting or near starting position. These self-movements cause large momentary changes in TTL and may appear to be an NPMA. Although SOs can cause momentary large shifts from the patient's weight in the closed system, appearing to break it, the closed system is not broken if the response of the system through the entire duration of the motion is considered. The patient movements (LPM or SO) are classified as slight movements or major movements and stored in a database in the memory device 64. If the patient movement amplitude comprises a postural change for a period of about 30 seconds, it is classified as a major movement. If the patient movement amplitude does comprises a postural change for a period of about 30 seconds, it is classified as a slight movement. These patient movements may be displayed on display screens 140, 150 on the user interface 54, as illustrated in FIG. 5.

In one embodiment, if patient movement and non-patient movement is detected at the same time point, both may be plotted simultaneously in the motion event space, as shown in FIG. 6. If the contribution of the non-patient movement motion is about a certain threshold such as about 20% or more of the total motion at a given time-point, the motion at that given time may be deemed a nurse assisted motion. In some embodiments, this threshold may be determined by the caregiver.

The user interface 54 illustrated in FIG. 5 includes a smart turn indicator. The smart turn indicator may comprise a guideline 152 that illustrates how long the nurse or caregiver has to reposition the patient again, depending on how long it has been since the last turn and the turning protocol 144. In the illustrated embodiment, the turning protocol 144 is to reposition the patient every 2 hours as shown on the first display 140. In other embodiment, the number of hours until the next turn is configurable for all patient support apparatuses 10. In some other embodiments, the number of hours until the next turn is configurable each patient support apparatus 10 individually. In another embodiment, there is a mapping of the patient's movement that may change time between repositioning automatically by the controller 28 based on the data processed by the processor 62. In other embodiments, the background and/or other features identifying the current time as compared to the time between repositioning may be color coded and/or based on time remaining for violation of the turning protocol 144. In other embodiments, the current time is relationship to the time remaining of the violation of the turning protocol 144 is demarcated by a “you are here” line. In some other embodiments, the amount of time until the next repositioning as determined by the turning protocol 144 is also affected by the number of major movements and/or slight movements of the patient.

Although this disclosure refers to specific embodiments, it will be understood by those skilled in the art that various changes in form and detail may be made without departing from the subject matter set forth in the accompanying claims.

1. A patient support apparatus comprising:
a control system including a controller, the controller operable to detect and classify movement data, and
a user interface including a display, the display illustrating the patient movement data, wherein movement data comprises patient movement data and non-patient motion artifacts.

2. The user interface of claim 2, wherein the display comprises a first portion and a second portion.

3. The patient support apparatus of claim 2, wherein the patient movement data comprises patient mobility score based on patient movement amplitude and patient frequency.

4. The patient support apparatus of claim 2, wherein the patient movement data comprises slight movement or major movement.

5. The patient support apparatus of claim 3, wherein the patient mobility score is displayed on the user interface as a mobility trend-line on the first portion of the display.

6. The patient support apparatus of claim 4, wherein the patient movement data displayed on the user interface comprises identification of caregiver assisted motion on the second portion of the display.

7. The patient support apparatus of claim 6, wherein the caregiver assisted motion occurs when both non-patient motion artifact and patient movement occurs at the same time, and when the non-patient motion artifact comprises more than about 20% of the movement data at that time.

8. The patient support apparatus of claim 4, wherein the patient movement amplitude comprises slight movement when the patient movement does not comprise a postural change for a period of about 30 seconds.

9. The patient support apparatus of claim 4, wherein the patient movement amplitude comprises major movement when the patient movement comprises a postural change in a period of about 30 seconds.

10. A system comprising:
a patient support surface supporting a patient,
a controller operable to detect and classify patient movement, and
a user interface including a display, the display illustrating the patient movement data,
wherein movement data comprises patient movement data and non-patient motion artifacts.

11. The system of claim 10, wherein a caregiver inputs a turning protocol comprising intervening time between patient turns, and wherein the patient movement data displayed on the user interface comprises a color coded display of the current time in relationship to the turning protocol.

12. The system of claim 11, wherein the turning protocol is also affected by an amount of major movements or slight movements of the patient.

13. The system of claim 10, wherein the patient movement is monitored by one or more continuous sensors.

14. A method of displaying movement data collected from a support apparatus comprising an inflatable mattress, the method comprising the steps of:
monitoring signals from a plurality of load cells, the plurality of load cells supporting inflatable mattress,
using a controller to process the signals from the load cells to detect and classify movement data, and
displaying the movement data on a display on a user interface,
wherein the movement data comprises patient movement data and non-patient motion artifacts.

15. The method of claim 14, wherein the display comprises a first portion and a second portion.

16. The method of claim 15, wherein the patient movement data comprises patient mobility score based on patient movement amplitude and patient frequency.

17. The method of claim 16, wherein the patient mobility score is displayed on the user interface as a mobility trend-line on the first portion of the display.

18. The method of claim 16, wherein the patient movement amplitude comprises major movement when the patient movement comprises a postural change in a period of about 30 seconds.

19. The method of claim 16, wherein the patient movement amplitude comprises slight movement when the patient movement does not comprise a postural change for a period of about 30 seconds.

20. The method of claim 14, wherein the method further comprises a caregiver inputting a turning protocol comprising intervening time between patient turns, and wherein the patient movement data displayed on the user interface comprises a color coded display of the current time in relationship to the turning protocol.
